(19) United States
(12) Patent Application Publication
(10) Pub. No.: US 2019/0139217 A1
(43) Pub. Date: May 9, 2019

(54) System and Method of Capturing Subtle Emotional Behavior

(71) Applicant: Harold Szu, Alexandria, VA (US)

(72) Inventor: Harold Szu, Alexandria, VA (US)

(21) Appl. No.: 16/103,885

(22) Filed: Aug. 14, 2018

Related U.S. Application Data
(60) Provisional application No. 62/545,421, filed on Aug. 14, 2017.

(51) Int. Cl. G06T 7/00; A61B 5/024; A61B 5/16; A61B 5/00; G06Q 50/26

(52) U.S. Cl. CPC G06T 7/0012; A61B 5/02433; A61B 5/165; A61B 5/489; A61B 2503/12; G06Q 50/265; G06T 2207/30101; G06T 2207/20084; G06T 2207/30016; A61B 5/7267

(57) ABSTRACT

A system of determining emotional inclination includes imaging and analysis subsystems. The imaging subsystem is configured to detect a dynamic vein map of a subject, illuminate the vein map, and record imaging data of the vein map. The analysis subsystem is configured to receive the data, analyze the data, and interpret the subject's emotional inclination based on the analysis. A method of determining emotional inclination includes detecting a dynamic vein map of a subject, illuminating the vein map, recording imaging data of the vein map, receiving the data, analyzing the data, and interpreting the emotional inclination based on the analysis.

### CROSS-REFERENCE TO RELATED APPLICATION

This is related to, and claims priority from, U.S. Provisional Application for Patent No. 62/545,421, which was filed on Aug. 14, 2017, the entire disclosure of which is incorporated herein by this reference.

### FIELD OF THE INVENTION

The invention relates to systems and methods of covertly determining the mindset and intentions of a subject using sensors and techniques applied, for example, through the use of a cellular telephone.

### BACKGROUND OF THE INVENTION

Crimes committed that relate to the mental state of the criminal, such as suicide bombings and other terrorist acts, are on the rise to the extent that they have become a usual aspect of modern life. Various techniques are used to deal with the commission of such acts and to minimize damage while such acts are taking place, but efforts are also made to prevent these actions before they occur. For example, identifying behavior of individuals that indicate preparation for committing a suicide bombing so that patterns of behavior can be detected can lead to increased scrutiny of a particular person to thwart any violent crime before anyone is hurt.

The historical perspective of suicide terrorism reaches back to long ago. For example, see FIG. 1. As shown in FIG. 1A, world-wide suicide terrorist causalities have increased dramatically in recent years, beginning over seven decades ago during WWII when Imperial Japan Kamikaze pilots attacked the USS Bunker Hill (FIG. 1B). More recently, sixteen suicide terrorists on Sep. 11, 2001 flew United Airlines flight 175 into the World Trade Center, killing 2996 people from over 90 countries, including 344 firefighters and 71 police officers (FIG. 1C). Suicide terrorists are no longer limited to men and children, and come from all reaches of the population (FIG. 1D).

Terrorism happens everywhere. For example, a cafe in Paris suffered casualties and recent incidents happened three times in London. It is likely that these events and occurrences in the U.S (Columbine High School, Virginia Tech, Sandy Hook Elementary School) were driven by personal and psychological causes, and not necessarily by political, social, or religious causes.

Violent suicidal behavior begins physically with a smaller Amygdale, and a lack of empathy for others and negative feelings towards oneself. This feeling of being “hope-less, help-less, worth-less,” escalates the “LESS triangle loop psychology,” which can be further compounded with ridiculous rationalism taking place at the Hippocampus due to such factors as political ideology, religion beliefs, or other belief systems, in order to involuntarily sacrifice other, innocent, people. FIG. 2 is an illustration showing a negative loop of a “LESS” Triangle, which can escalate into Suicidal Psychology.

This behavior is different than that of a terrorist who has been trained for the purpose of causing violence, even at the cost of his or her own death. Surveillance by intelligence agencies (as well as police forces and Homeland Security) and observation of patterns of behavior can lead to early prevention of a terrorist act by one who has trained and prepared for committing the act, especially as a scheduled event. The suicide terrorist who acts as the result of a mental disturbance is much more unpredictable. Such a person doesn't plan the act, or even know ahead of time that he or she will commit the act. In such a person, any type of action could trigger suicidal violent behavior, put in play by a mental disturbance that could have gone unnoticed even by close family members. Only discovery of physiological warning signs (engorged veins, subtle changes in vocal tone and facial expression) could indicate that this person might have gone over the edge and may soon act.

South Korea has had notable success in countering suicide bombings and terrorist acts, despite the nearby threatening adversary of North Korea. Possible reasons for this success could be (1) better K-12 education, (2) better living standard and job perspectives, (3) a populace that is sick and tired of five decades of senseless killing, and (4) strong preventive law enforcement training. As a result, terrorist acts that might have happened did not materialize in South Korea (certainly not big events in the world news).

FIG. 3 show a screen shot from a YouTube video in which the JTBC reported on a Korean Counter Terrorist Program, for training.

FIG. 4 shows that counter-terrorist wide-spread efforts begin with k-12 school counter-bullying efforts in Korea.

FIG. 5 shows that anti-riot and anti-bullying training employ martial arts for police forces, for both men and woman officers.

One reason South Korea has no suicide terrorists is because they consider life to be precious after a half-century of war followed by the reconstruction period, resulting in prosperity by means of heavy industrialization in steel ship manufacturing and electronics semiconductor chip DRAM fabrication, as well as communication industry growth enabled by Smartphone Information Technology. There are also plenty of lower-end labor market jobs, which are available for the North Korean people working either legally in the demilitarized zone or illegally in Seoul. Useful war experience has been transformed into peacetime first-class police training, providing police officers of both genders as well as martial artists with keen observation and sensitivity. Referring to FIG. 6, persistent surveillance in daily training of law enforcement is perhaps a key remediation to counter terrorists.

It would therefore be beneficial to be preventive and pre-emptive regarding suicide terrorists. Extending the applications of artificial intelligence, use of Unified Deep Learning Machine Learning to capture the intuition and hunch of potential suicide terrorists by those experienced in law enforcement would be such a pre-emptive measure. Therefore, using current ubiquitous technology to apply this extension to capture, using bionic smart sensors pairs (such as for hawk eyes, cat ears, and dog noses) and to develop training data to be further down-selected such as by Korea Law Enforcement would be a huge step in the fight against suicide terrorism.

### BRIEF SUMMARY OF THE INVENTION

According to an aspect of the invention, a system of determining emotional inclination includes an imaging subsystem and an analysis subsystem. The imaging subsystem is configured to detect a dynamic vein map of a subject, to actively illuminate the dynamic vein map, and to record imaging data of the dynamic vein map. The analysis subsystem is configured to receive the recorded imaging data, to analyze the recorded imaging data, and to interpret the subject's emotional inclination based on the analysis.

For example, the imaging subsystem can be housed in a portable electronic device, such as a cellular telephone. Alternatively, the imaging subsystem can include, or be included as a component of, a larger imaging system, such as a body scanning device.

The imaging subsystem can include short-wave infrared image capture and processing circuitry, and can include a near-infrared filter passive camera and/or a 0.8-2 micron digital video imaging camera. The imaging subsystem can be fabricated as a microelectromechanical system.

The subject's emotional inclination can be, for example, the subject's brain internal state.

The analysis subsystem can include an artificial neural network, which can be configured to apply a deep learning algorithm.

According to another aspect of the invention, a method of determining emotional inclination includes detecting a dynamic vein map of a subject, actively illuminating the dynamic vein map, recording imaging data of the dynamic vein map, receiving the recorded imaging data, analyzing the recorded imaging data, and interpreting the subject's emotional inclination based on the analysis.

The method can also include housing the imaging subsystem in a portable electronic device, such as a cellular telephone. The method can also include providing the imaging subsystem as a component of a body scanning device.

The method can also include providing the imaging subsystem with short-wave infrared image capture and processing circuitry, with a near-infrared filter passive camera, and/or with a 0.8-2 micron digital video imaging camera. The imaging subsystem can be fabricated as a microelectromechanical system.

The subject's emotional inclination can be, for example, the subject's brain internal state.

The method can also include providing the analysis subsystem with an artificial neural network, which can be configured to apply a deep learning algorithm.

### BRIEF DESCRIPTION OF THE DRAWINGS

FIG. 1A is a graph showing the increase in world-wide suicide terrorist causalities over time.

FIG. 1B is a photo of the USS Bunker Hill after an Imperial Japanese Kamikaze attack during WWII.

FIG. 1C is a photo of the World Trade Center in NYC after the 9/11 suicide terror attack.

FIG. 1D is a photo of a present-day suicide terrorist.

FIG. 2 is an illustration showing a negative loop of a “LESS” Triangle.

FIG. 3 show a screen shot from a YouTube video related to a Korean Counter Terrorist Program.

FIG. 4 shows a publication regarding k-12 school counter-bullying efforts in Korea.

FIG. 5 illustrates training including martial arts for police forces.

FIG. 6 illustrates persistent surveillance in daily training of law enforcement.

FIG. 7A is an illustration of a smartphone in which SWIR Video Imaging Technology is installed.

FIG. 7B is an illustration of a SWIR CMOS component.

FIG. 7C shows an exemplary MEMS and day video camera of a smartphone.

FIG. 7D shows an exemplary long-distance passive SWIR image.

FIG. 8 shows an equivalent day photo for facial stress-popping vein dynamics.

FIG. 9A shows an image of a brain as viewed from the underside and front.

FIG. 9B shows an image of a brain with the Thalamus and Corpus Striatum (Putamen, caudate, and Amygdale) splayed out to show detail.

FIG. 10A is an illustration of comprehensive electromagnetic spectra for sensors.

FIG. 10B is an illustration of passive sub-millemeter wave imaging used in airports.

FIG. 10C is an illustration of TeraHz experiments.

FIG. 10D is an illustration of a vein map.

FIG. 10E is an illustration of image processing for facial stress vein popping.

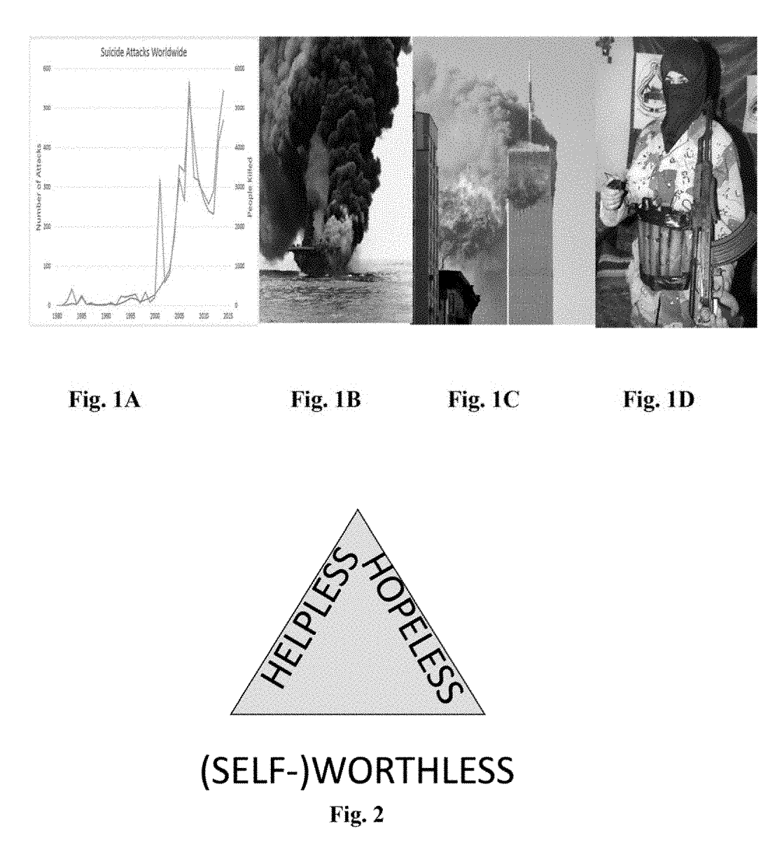

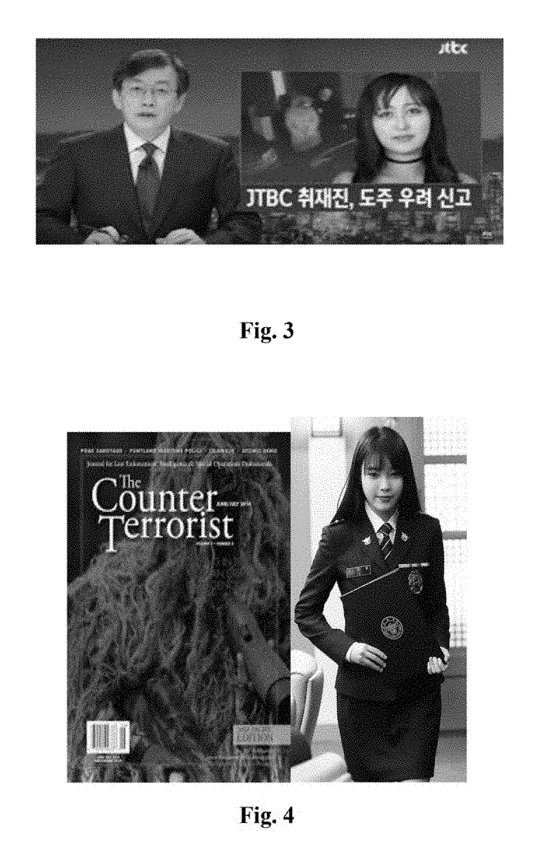

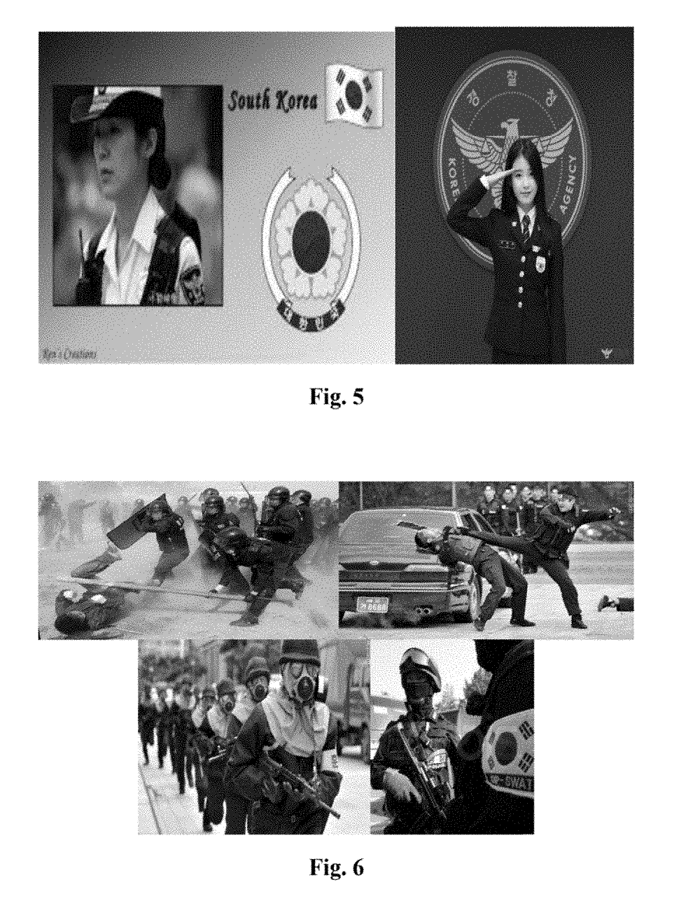

### DETAILED DESCRIPTION OF THE INVENTION

A terrorist will have an increased heart beat, driving hot bubbling blood to circulate through the face, head, and core body, physiologically revealing that the potential terrorist might initiate suicide detonation without yet knowing his or her own intention. This can be detected in advance by a Smartphone Active Short Wave Infrared (SWIR) Video taken of a Dynamic Vein Map (DVM) computed using a Deep Learning Algorithm in real-time phase transition. From the sensory consideration in the design, a Near-infrared (NIR) Passive Filter or SWIR active imaging capability, costing about $50˜$300, is installed in a Smartphone MEMS platform near day video that can covertly track the facial DVM of a user at a safe distance.

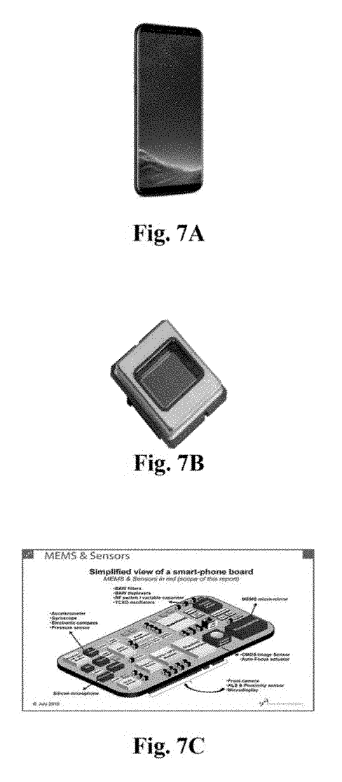

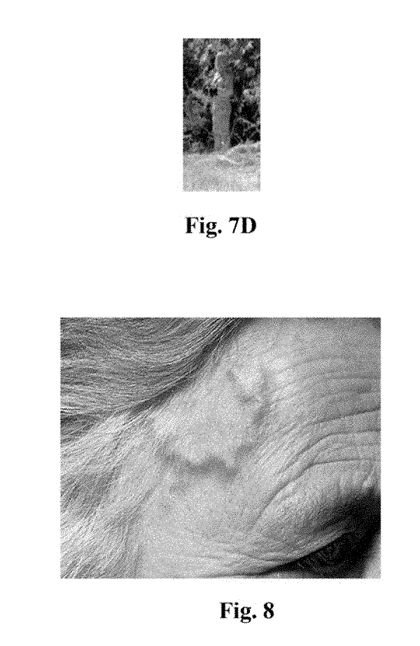

AI ANN (artificial neural network) machine learning is applied to detect Suicidal Terrorist behavior ahead of a violent act. The complexity of such a subtle emotional response forms a class of cohort biometrics involving IQ, e-IQ, culture, religion, and belief. Biologically, a relatively retarded Hippocampus for associative memory IQ and small Amygdale sizes for low social skill e-IQ are attributes indicating Suicidal Terrorist behavior. A Smartphone, with a Day and Night Video SWIR 0.8-2 micron Digital Video Imaging Camera are indicated in FIG. 7.

FIG. 7A is a depiction of SWIR Video imaging Technology as installed within a smartphone. FIG. 7B is an illustration of a SWIR CMOS component; such a component can provide 60 frames per second full frame rate in a 1920×1080 pixel format with 10 μm pitch, with the capability for 100% duty cycle across the entire illumination intensity range. Preferably, it has a high sensitivity in the 0.9 to 1.7 μm spectrum, NIR/SWIR, from 0.7 to 1.7 μm, VIS/SWIR from 0.5 to 1.7 μm (optional), with a digital 12-bit output and operation from −40 to +70° C. FIG. 7C shows an exemplary MEMS and day video camera of a smartphone. FIG. 7D shows an example of a long-distance passive SWIR image.

A DVM is actively illuminated in a near-infrared image SWIR video that is not visible to the human visual system and that is possibly also covert to the potential terrorist. FIG. 8 shows an equivalent day picture for facial stress-popping vein dynamics. A Computational Intelligence design can be used to help law enforcement personnel using a smartphone to catch the Brain Internal State (BIS) of a Suicide Bomber or Terrorist a few minutes early (or earlier), in order to prevent senseless killing and someday to eradicate the suicide terror pandemic entirely. Smartphones are loaded with a Deep Learning Algorithm (DLA) together with a passive Near IR filter (R 72) or active Short Wave Infrared 0.8-2 μm illumination.

Deep Learning implies multiple layers of neural networks for multiple feature extraction to increase the probability of detection of overly stressed emotion intelligence, and to reduce the false alarm rate. This is similar to the biological neural network (BNN) of the Human Visual System (HVS) in the back of head Cortex 17 area, from V1 layer to V4 layer. While a false positive is a nuisance, a false negative is detrimental to innocent bystanders. Results of studies favor passive NIR using Filter R70 or an active SWIR. The DVM tells the detonation exit time behavior of a terrorist. In order to measure the stress e-IQ for preemptive action, mood and temper change can be tracked by illuminating the subject from a distance with SWIR or passive near infrared (0.8 m) light penetration processing pseudo-real time video recording.

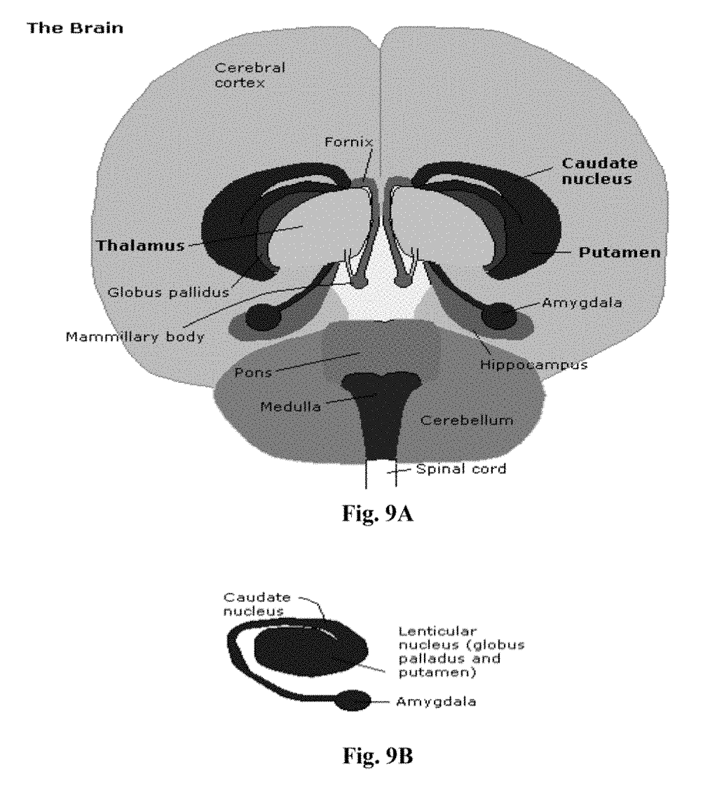

FIG. 9 shows an image of a brain as viewed from the underside and front (FIG. 9A). The Thalamus and Corpus Striatum (Putamen, caudate, and Amygdale) are splayed out to show detail (FIG. 9B). The BIS might be caused by abnormal brain anatomy (ABA); for example, a smaller Amygdale can lead to negative feelings of “low self-worth, hopelessness, and helplessness” when compounded with retarded Hippocampus Associative Memory. The brain can easily be washed with self-justified terrorism, that is, can illogically drive the subject to commit suicidal acts of terror. Homo-sapiens' emotional center is located at the two Amygdale (in Latin: almond shape) in the brain Limbic system that can activate the Sympathetic Nervous System that flood the body with stress hormone, which can lead to acute phobias. The size of the Amygdala is critical to the development of social skills, is responsible for the “fight or flight” response, and, in the extreme, can cause suicidal intentions.

Health is a prerequisite of happiness. A larger Amygdale enables a greater societal integration and cooperation with others and increases the level of a person's emotional intelligence. The key is to develop healthy Amygdale riding on Hippocampus Associative Memory, which is critical for a person's healthy psychology. When one is young, scouting team work is good training. When one is encouraged to participate in sports activities, negative feelings of self-depreciation can be released.

Salience is necessary to avoid over-fitting or lacking of depth of field. Some spectra do not propagate far in the air. See-through cloth with two separated polarizations at either at PMMW, Terra Hz (sub mm wave), or Police Speed Gun, DHS Body Scan using Passive Millimeter Wave 3 mm wave (80 GHz-100 GHz) which like radiometer reads passive infrared heat radiation occluded by solid metal object then it penetrates through the cloth to the camera. Terrorist Cohort Biometrics “You don't have it (sensed), you can't get it” no matter how powerful AI ANN Deep Learning is. Nothing can do the magic, unless you have all the salient features measured—by smart power of pairs of eyes, ears, nostrils, etc.

The gathered data will be further down-selected by seasoned law enforcement to avoid over-fitting or missing degrees of freedom (d.o.f.). The design architecture will include layers of ANN, the shape of hidden layers will be selected (hour glass (condensation)), or beer barrel (integration)), and dynamic learning of the architecture will take place. Test and evaluation will take place in the lab and in the field.

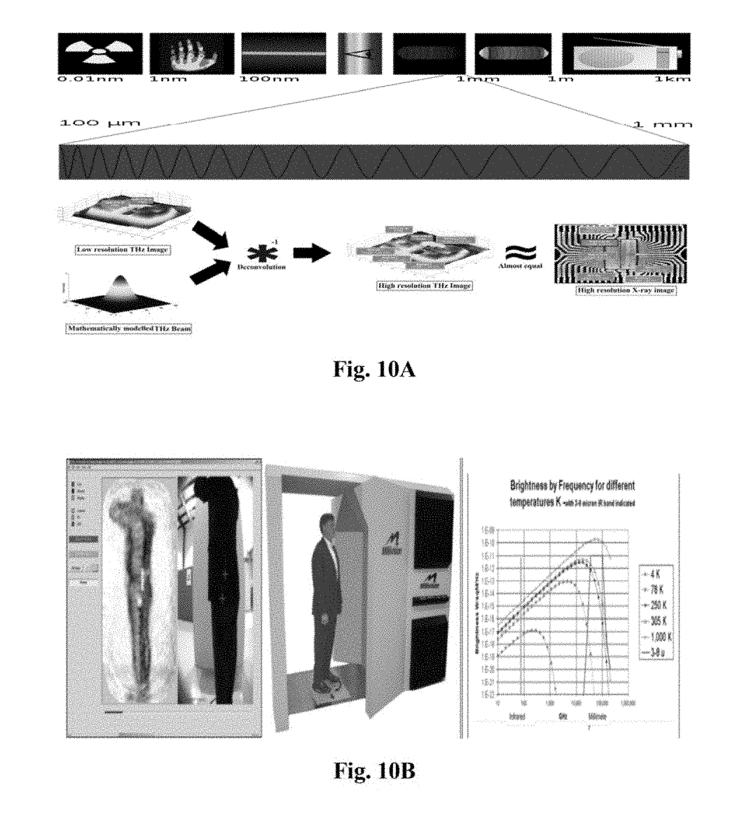

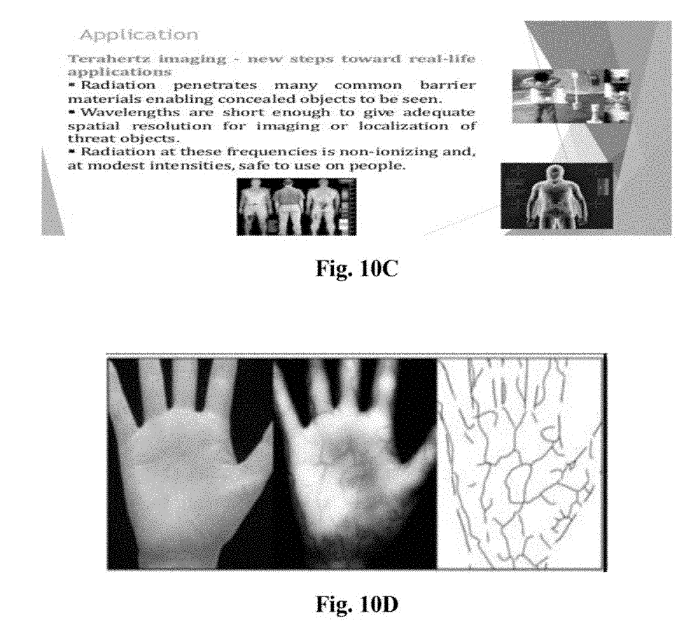

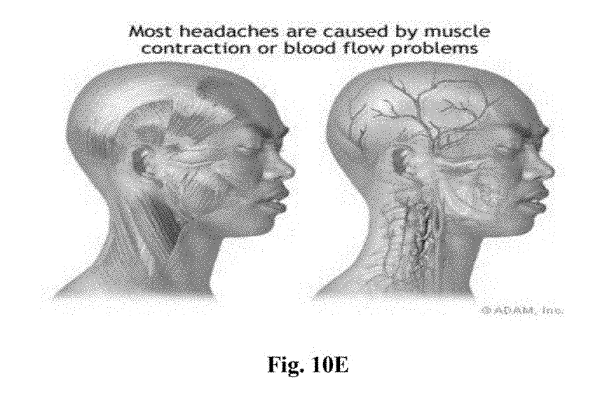

FIG. 10A shows comprehensive electromagnetic spectra for sensors; FIG. 10B shows passive sub-millemeter wave (PMMW) used in airports; FIG. 10C shows TeraHz experiments; FIG. 10D shows Japanese company developed vein map; and FIG. 10E shows image processing for facial stress vein popping.

Studies show that SWIR is favored based on cost, ease of use, and portability, to provide a Dynamic Vein Map (DVM) that tells the detonation exit time behavior of a potential terrorist. To measure stress e-IQ for preemptive ST, mood/temper change can be tracked by dynamic illumination using near infrared (0.8 m) light penetration processing real time video recording. A medical static contact vein map can be extended as a biometric ID using ultrasound, and NIRAI Expert System Logic is simply a set of programming logic based on

IF . . .

Then . . .

Return. (1)

ANN begins with a data Vector Time Series for Power of Pairs [2]:

X̅ₚₐᵢᵣₛ(t)=[Aᵢⱼ]S̅ₚₐᵢᵣₛ(t) (2)

And the inverse is solved using a Convolution Neural Network:

Ŝₚₐᵢᵣₛ(t)=[Wⱼᵢ(t)]X̅ₚₐᵢᵣₛ(t) (3)

ANN are derived from Natural Intelligence based on a constant brain temperature at 37° C., where the disagreement noise of pairs of eyes and ears will decay rapidly to thermal equilibrium.

$$\begin{matrix} Theorem 1 : Constant Temperature Brain & \\ {S}_{tot} = {k}_{B} Log {W}_{MB} & ( 4 ) \\ \begin{matrix}{W}_{MB} = exp ( \frac{{S}_{tot}}{{k}_{B}} ) \\ = exp ( \frac{( {S}_{brain} + {S}_{env .} ) {T}_{o}}{{k}_{B} {T}_{o}} ) \\ = exp ( \frac{{S}_{brain} {T}_{o} - {E}_{brain}}{{k}_{B} {T}_{o}} ) \\ = exp ( - \frac{{H}_{brain}}{{k}_{B} {T}_{o}} )\end{matrix} & ( 5 ) \\ & \\ \begin{matrix} Δ {S}_{tot} > 0 \\ NI is based on Boltzmann : Δ {H}_{brain} = Δ {E}_{brain} - {T}_{o} Δ {S}_{brain} \leq 0 , \\ because of irreversible Δ {S}_{brain} > 0\end{matrix} & ( 6 ) \\ Lyaponov : \frac{Δ {H}_{brain}}{Δ t} = ( \frac{Δ {H}_{brain}}{Δ [ {W}_{i , j} ]} ) \frac{Δ [ {W}_{i , j} ]}{Δ t} = - \frac{Δ [ {W}_{i , j} ]}{Δ t} \frac{Δ [ {W}_{i , j} ]}{Δ t} = - {( \frac{Δ [ {W}_{i , j} ]}{Δ t} )}^{2} \leq 0 & ( 7 ) \\ Newton : \frac{Δ [ {W}_{i , j} ]}{Δ t} = - \frac{Δ {H}_{brain}}{Δ [ {W}_{i , j} ]} & ( 8 ) \\ Hebb : \frac{Δ [ {W}_{i , j} ]}{Δ t} ≡ - \frac{Δ {H}_{brain}}{Δ [ {W}_{i , j} ]} = ( - \frac{Δ {H}_{brain}}{Δ {\vec{Dendrite}}_{j}} ) \frac{Δ {\vec{Dendrite}}_{j}}{Δ [ {W}_{i , j} ]} ≡ {\vec{g}}_{j} {\vec{S}}_{i} & ( 9 ) \\ Dendrite input : {D}_{i} ≡ {\sum }_{k} [ {W}_{i , k} ] {S}_{k} & ( 10 ) \\ Gilal Cells : {\vec{g}}_{j} ≡ ( - \frac{Δ {H}_{brain}}{Δ {\vec{Dendrite}}_{j}} ) ; & ( 11 ) \\ Sigmoid Threshold Neuron : {S}_{i} = σ ( {\sum }_{j = X 1}^{X 2} {W}_{ij} {X}_{j} - {θ}_{i} ) \geq 0 ; & ( 12 )\end{matrix}$$

$$\begin{matrix} Theorem 2 : Unified Deep Learning \\ From Therorem 1 of MFE follows the Glail Cells definition & \\ {g}_{j} ≡ - \frac{\partial {H}_{brain}}{\partial {dendrite}_{j}} = - \frac{\partial {H}_{brain}}{\partial {S}_{j}} \frac{\partial {S}_{j}}{\partial {dendrite}_{j}} = - \frac{\partial {H}_{brain}}{\partial {S}_{j}} {σ}_{j}^{( i )} ( {dendrite}_{j} ) \\ where & ( 13 ) \\ - \frac{\partial {H}_{brain}}{\partial {S}_{j}} = - {\sum }_{k} \frac{\partial {H}_{brain}}{\partial {dendrite}_{k}} \frac{\partial {dendrite}_{k}}{\partial {S}_{j}} - {\sum }_{k} \frac{\partial {H}_{brain}}{\partial {dendrite}_{k}} \frac{\partial }{\partial {S}_{j}} {\sum }_{i} [ {W}_{k , l} ] {S}_{i} = {\sum }_{k} {\tilde{g}}_{k} [ {W}_{k , j} ] & ( 14 ) \\ {g}_{j} = {σ}_{j}^{( i )} ( {dendrite}_{j} ) {\sum }_{k} {\tilde{g}}_{k} [ {W}_{k , j} ] & ( 15 )\end{matrix}$$

Both Supervised Deep Learning (SDL) and Unsupervised Deep Learning (UDL) are self-similarly derived within the derivative of the sigmoid window function.

σⱼ⁽ⁱ⁾(dendriteⱼ); σⱼ⁽ⁱ⁾(netⱼ): O(Δt)=η in terms of the backward error propagation algorithms are isomorphic:

$$[ {W}_{ji} ( t + 1 ) ] - [ {W}_{ji} ( t ) ] = \{ \begin{matrix}η {\tilde{S}}_{i} {σ}_{j} ( {dendrite}_{j} ) \{ 1 - {σ}_{j} ( dendrite ) \} {\sum }_{k} {\tilde{g}}_{k} [ {W}_{k , j} ] + {α}_{momtum} [ {W}_{ji} ( t ) - [ {W}_{ji} ( t - 1 ) ] ] \\ η {\vec{S}}_{i} {σ}_{j} ( {net}_{j} ) \{ 1 - {σ}_{j} ( {net}_{j} ) \} {\sum }_{k} {\tilde{δ}}_{k} [ {W}_{k , j} ] + {α}_{momtum} [ {W}_{ji} ( t ) - [ {W}_{ji} ( t ) - [ {W}_{ji} ( t - 1 ) ] ]\end{matrix} \} \qquad ( 16 a , b )$$

Capturing the expert experience of Korean law enforcement into AI logic in terms of a set of dynamic feature vectors will allow the powerful real-time computational platform of the current Smartphone to process the deep learning to provide a decision aid to users world-wide.

### REFERENCES

For background purposes, the substance of the following references is incorporated herein.
[1] Korean Emotional Intelligence Project, KAIST PI: Prof. Soo-Yung Lee, Brain Science Research Center, Center for Artificial Intelligence Research, Joint R&D Center for Brain Science and Technology Applications, ITC B/D(N1) #512, KAIST 291 Daehak-ro, Yuseong-gu, Daejeon 34141, Republic of Korea
[2] Harold Szu, Mike Wardlaw, Jeff Willey, Charles Hsu, Kim Scheff, Simon Foo, Henry Chy, Joseph Landa, Yufeng Zheng, Jerry Wu, Eric Wu, Hong Yu, Guna Seetharaman, Jae H. Cha, John E. Gray, “Theory of Glial Cells & Neurons Emulating BNN for N1 operated effortlessly at MFE,” MedCrave Online J. (MOJ) Appl. Bionics Biomechanics (ABB). May 18, 2017pp. 1-26.
[3] “Learning Machine,” Nicola Jones Nature V. 505, pp146-148, 2014;
[4] “Deep Learning,” Yann LeCun, Yosbui Bengio, Geoffrey Hinton, Nature V. 521, pp. 436-440, 2015.
[5] “Natural Intelligence Neromorphic Engineering,” Harold Szu, Elsevier 2017, pp. 1-350.
[6] “Harold Szu, Lidan Miao, Hairong Qi, Unsupervised Learning at MFE” Proc. SPIE Vol. 6576, p. 657605, (2007)
[7] Multiple Layer Deep Learning appeared in “Introduction to Computing with Neural Nets,” Richard Lipmann, IEEE ASSP Magazine April 1987; PDP Book, (MIT Press 1986 book by James McCelland, David Rumelhart, PDP group); Paul Werbos, Ph. D. Thesis, Harvard U.
[8] Eric Newman, “New roles for Astrocytes: Regulation of Synaptic transmission,” Trends in Neuroscience, Vol. 26, No. 10, 2003.
[9] Douglas Fields, and Beth Steven-Graham, “New Insights into Neuron-Glia Communication,” pp. 556-562. SCIENCE, Vol. 18, 2002.

I claim:

1. A system of determining emotional inclination, comprising:
an imaging subsystem configured to detect a dynamic vein map of a subject, to actively illuminate the dynamic vein map, and to record imaging data of the dynamic vein map; and
an analysis subsystem configured to receive the recorded imaging data, to analyze the recorded imaging data, and to interpret the subject's emotional inclination based on the analysis.

2. The system of claim 1, wherein the imaging subsystem is housed in a portable electronic device.

3. The system of claim 2, wherein the portable electronic device is a cellular telephone.

4. The system of claim 1, wherein the imaging subsystem includes a body scanning device.

5. The system of claim 1, wherein the imaging subsystem is included as a component of a body scanning device.

6. The system of claim 1, wherein the imaging subsystem includes short-wave infrared image capture and processing circuitry.

7. The system of claim 6, wherein the imaging subsystem includes a 0.8-2 micron digital video imaging camera.

8. The system of claim 1, wherein the imaging subsystem includes a near-infrared filter passive camera.

9. The system of claim 1, wherein the imaging subsystem is fabricated as a microelectromechanical system.

10. The system of claim 1, wherein the subject's emotional inclination is the subject's brain internal state.

11. The system of claim 1, wherein the analysis subsystem includes an artificial neural network.

12. The system of claim 11, wherein the artificial neural network is configured to apply a deep learning algorithm.

13. A method of determining emotional inclination, comprising:
detecting a dynamic vein map of a subject;
actively illuminating the dynamic vein map;
recording imaging data of the dynamic vein map;
receiving the recorded imaging data;
analyzing the recorded imaging data; and
interpreting the subject's emotional inclination based on the analysis.

14. The method of claim 13, further comprising housing the imaging subsystem in a portable electronic device.

15. The method of claim 14, wherein the portable electronic device is a cellular telephone.

16. The method of claim 13, further comprising providing the imaging subsystem as a component of a body scanning device.

17. The method of claim 13, further comprising providing the imaging subsystem with short-wave infrared image capture and processing circuitry.

18. The method of claim 17, further comprising providing the imaging subsystem with a 0.8-2 micron digital video imaging camera.

19. The method of claim 13, further comprising providing the imaging subsystem with a near-infrared filter passive camera.

20. The method of claim 13, further comprising fabricating the imaging subsystem as a microelectromechanical system.

21. The method of claim 13, wherein the subject's emotional inclination is the subject's brain internal state.

22. The method of claim 13, further comprising providing the analysis subsystem with an artificial neural network.

23. The method of claim 22, further comprising configuring the artificial neural network to apply a deep learning algorithm.
